(19) United States
(12) Patent Application Publication
(10) Pub. No.: US 2009/0131987 A1
(43) Pub. Date: May 21, 2009

(54) BONE REDUCTION DEVICE AND METHOD UTILIZING SAME

(75) Inventor: Amir M. MATITYAHU, Los Altos, CA (US)

Correspondence Address: Dorsey & Whitney LLP;US Bank Center, 1420 Fifth Avenue, Suite 3400, Seattle, WA, 98101-4010, US

(73) Assignee: ANTHEM ORTHOPAEDICS LLC, Los Altos, CA (US)

(21) Appl. No.: 12/233,570

(22) Filed: Sep. 18, 2008

Related U.S. Application Data
(60) Provisional application No. 60/973,353, filed on Sep. 18, 2007.

(51) Int. Cl. A61B 17/70

(52) U.S. Cl. 606/280; 606/282

(57) ABSTRACT

A bone reduction device for use with a bone plate includes a plate attachment arm having at least one aperture for receiving at least one plate attachment device of the bone plate for coupling the plate attachment arm to the shaft of the bone plate. A bone attachment arm is disposed alongside the plate attachment device and has at least one aperture for receiving at least one bone attachment device of the bone plate for coupling the bone attachment arm to a segment of a bone. The bone attachment arm is moveable relative to the plate attachment arm. An adjustment mechanism is carried by the plate attachment arm and engages the bone attachment arm for translating the bone attachment arm relative to the plate attachment arm.

### CROSS-REFERENCE TO RELATED APPLICATIONS

This application claims the benefit of related U.S. Provisional Patent Application Ser. No. 60/973,353 filed Sep. 18, 2007, the entire content of which is hereby incorporated by this reference.

### SCOPE OF THE INVENTION

The present invention relates to a device and method for reducing a segmented bone.

### BACKGROUND

Current methods of reducing segmented bones, such as fractured or osteotomised bones in a mammalian body, involve moving or reducing a portion of the bone, generally the distal portion of the bone, either by grasping the bone portion with a human hand or clamp or attaching a pin to the bone portion and grasping the pin with a hand to move the bone portion. These methods often include making an incision through the skin of the patient and then drilling into the bone, then moving the bone portion by hand into position and holding the bone portion once positioned. Other methods may include a large incision in the tissue surrounding the bone for viewing and manual alignment of the bone portion, as well as manual affixation of the separated bone portions. Unfortunately, such methods are imprecise and are often physically hard on the patient and the surgeon. Other current methods include utilization of a screw advanced through a targeting jig and bone plate into the bone portion, whereby rotation of the screw in the bone portion cause the bone portion to be drawn toward the bone plate, namely, causing the bone portion to move relative to the bone plate in a direction generally perpendicular to the plate, so as to reposition the bone portion. Unfortunately, such screws often can not move the bone to the plate due to the position of the bone or move in an oblique fashion causing the screw to bend and, on occasion, break.

What is needed in the art is an apparatus and assembly for repositioning portions of a segmented bone to permit securement of the bone to a bone plate or other corrective device in a measured and precise manner.

### SUMMARY OF THE INVENTION

A bone reduction device for use with a bone plate is provided and includes a plate attachment arm having at least one aperture for receiving at least one plate attachment device of the bone plate for coupling the plate attachment arm to the shaft of the bone plate. A bone attachment arm is disposed alongside the plate attachment device and has at least one aperture for receiving at least one bone attachment device of the bone plate for coupling the bone attachment arm to a segment of a bone. The bone attachment arm is moveable relative to the plate attachment arm. An adjustment mechanism is carried by the plate attachment arm and engages the bone attachment arm for translating the bone attachment arm relative to the plate attachment arm.

### BRIEF DESCRIPTION OF THE DRAWINGS

FIG. 1 is a perspective view of a bone reduction device according to an embodiment of the present invention attached to a bone plate and showing a tibia broken into spaced apart first and second bone segments.

FIG. 2 is a perspective view of the bone reduction device of FIG. 1 showing the first and second bone segments reduced and aligned.

FIG. 3 is a perspective view of the bone reduction device of FIG. 1, taken from line 3-3 of FIG. 1.

FIG. 4 is a perspective view of the bone reduction device of FIG. 1, taken from line 4-4 of FIG. 2.

FIG. 5 is an end elevation view of the bone reduction device, bone plate and bone of FIG. 1 taken along line 5-5 of FIG. 2.

FIG. 6 is an end elevation view of the bone reduction device, bone plate and bone of FIG. 1 taken along line 6-6 of FIG. 2.

FIG. 7 is a side elevation view of the bone reduction device, bone plate and bone of FIG. 1 taken along line 7-7 of FIG. 4.

FIG. 8 is a side elevation view of the bone reduction device, bone plate and bone of FIG. 1 taken along line 8-8 of FIG. 4.

FIG. 9 is a perspective view of the bone reduction device of FIG. 1, having attachment screws inserted into bone.

FIG. 10 is an exploded perspective view of the bone reduction device of FIG. 1.

FIG. 11 is an elevation view of a bone reduction device according to an alternative embodiment attached to a bone plate and bone.

FIG. 12 is a cut-away perspective view of the bone reduction device, bone plate and bone of FIG. 11, absent the plate attachment device(s) or peg(s).

FIG. 13 is a perspective view of the bone reduction device, bone plate and bone of FIG. 11, taken along line 13-13 of FIG. 12, but showing the plate attachment devices or pegs inserted.

### DESCRIPTION OF THE INVENTION

The figures illustrate a bone reduction device 20 for moving fractured bone fragments or segments so as to align the segments in an anatomical position to heal. The bone reduction device is illustrated for use with a suitable bone such as a tibia 21 that has been broken, fractured or osteotomized between its proximal and its distal end into two or more bone segments. It is appreciated that the bone reduction device can be used on any bone in the mammalian body. Use in association with a bone which has been broken, fractured, or osteotomised into two segments is discussed below, although it is contemplated that more than two bone segments or fragments may be reduced or repaired using one or more bone reduction devices.

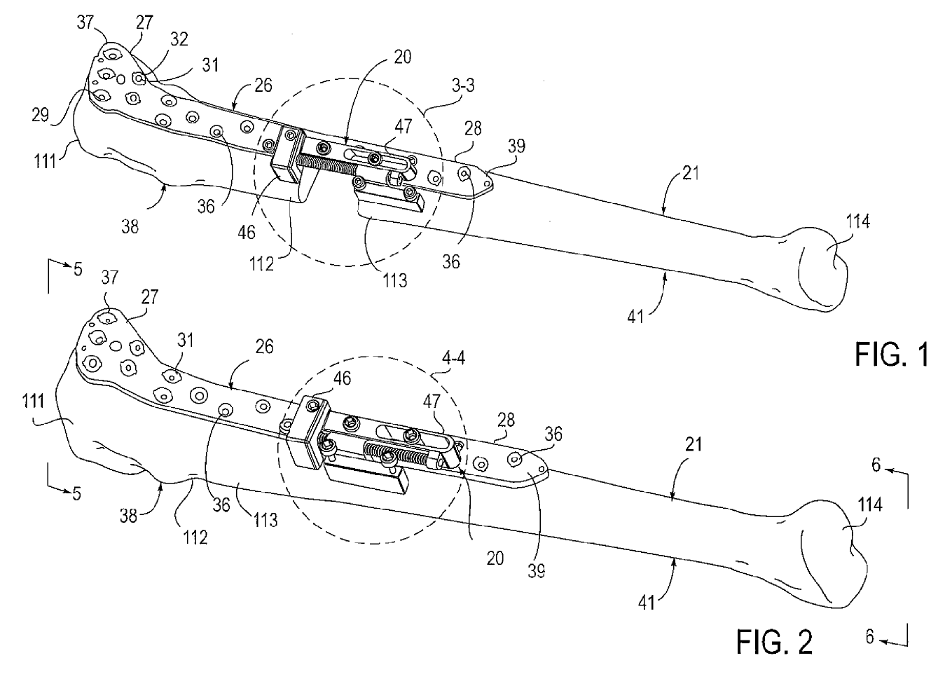

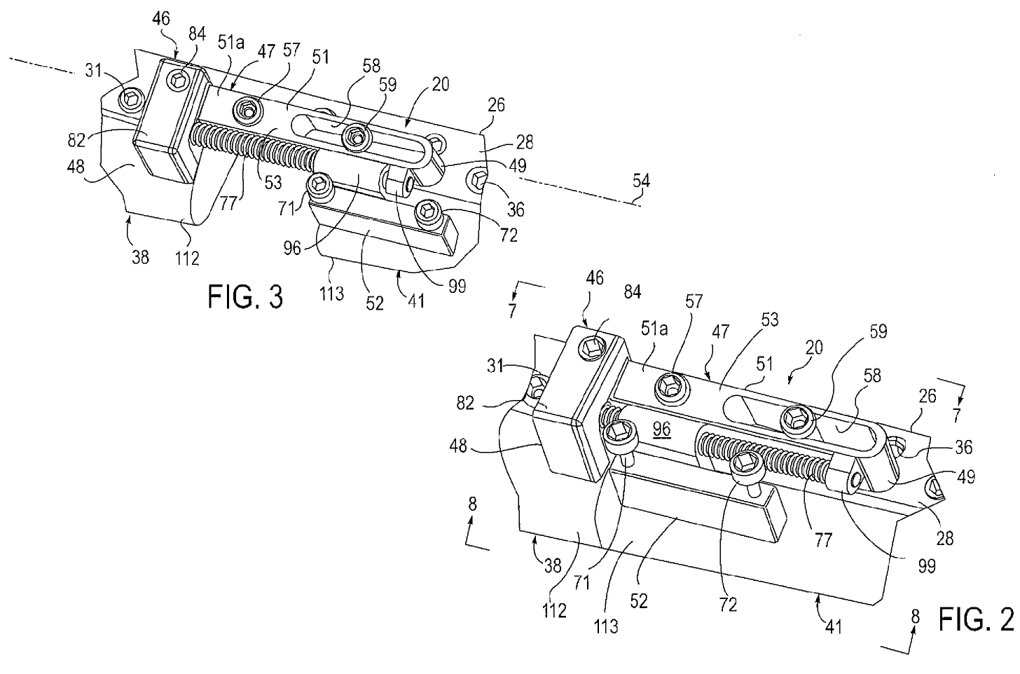

The bone reduction device 20 of the embodiment disclosed is for use with a bone plate, and preferably a locking bone plate 26. The bone plate or locking bone plate as seen in FIGS. 1-2 has a head 27 and a shaft 28, and includes a plurality of apertures 29 for receipt of one or more locking devices for screws 31 In a preferred embodiment, the bone plate includes one or more apertures 29 that receive a bushing 32 having an inner thread. The inner thread of the bushing mates with an outer thread on a screw, such as a bone screw 31. Additionally, one or more threaded apertures 36 may be provided in the bone plate itself. The bone plate may be attached to a targeting jig (not shown), which can be a generic jig currently available from many suppliers. The bone plate 26 in the illustrated embodiment has a first end or proximal or top end 37 which is secured, or locked, in position on a proximal bone segment 38. The bone plate is locked in position by one or more bone screws extending through apertures and/or bushings in the proximal end of the bone plate and into the bone 21. Shaft 28 extends distally of head 27 to a second end or distal end 39 of the bone plate 26.

In FIGS. 1 and 3, a first or proximal bone segment 38 is separated from a second or distal bone segment 41. The distal bone segment is spaced distally of the proximal bone segment. Operation of the bone reduction device of the present invention results in the distal bone segment 41 being translated proximally and aligned with the proximal bone segment 38, as shown in FIGS. 2 and 4, placing the segments in the proper anatomical position to heal. Once the broken or distal segment 41 is repositioned next to the proximal bone segment 38 and plate 26, the bone 21 will be aligned and straight.

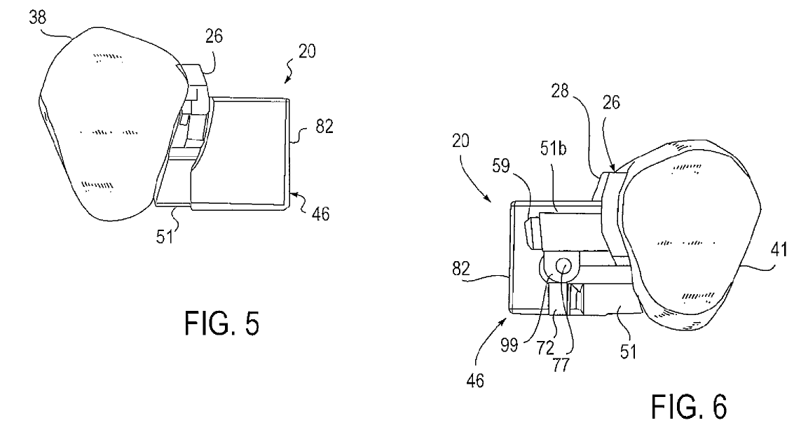

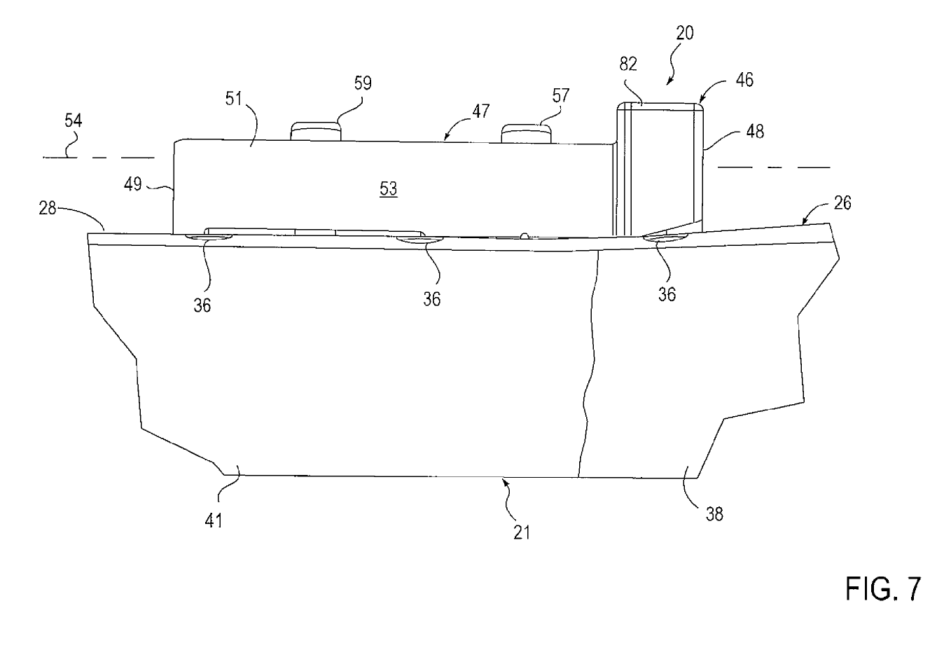

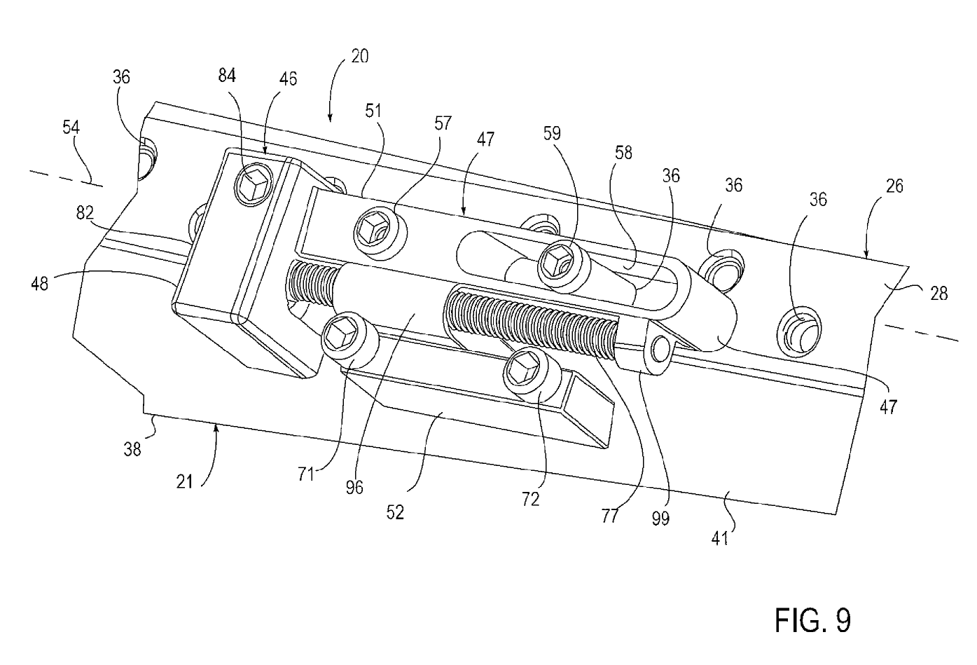

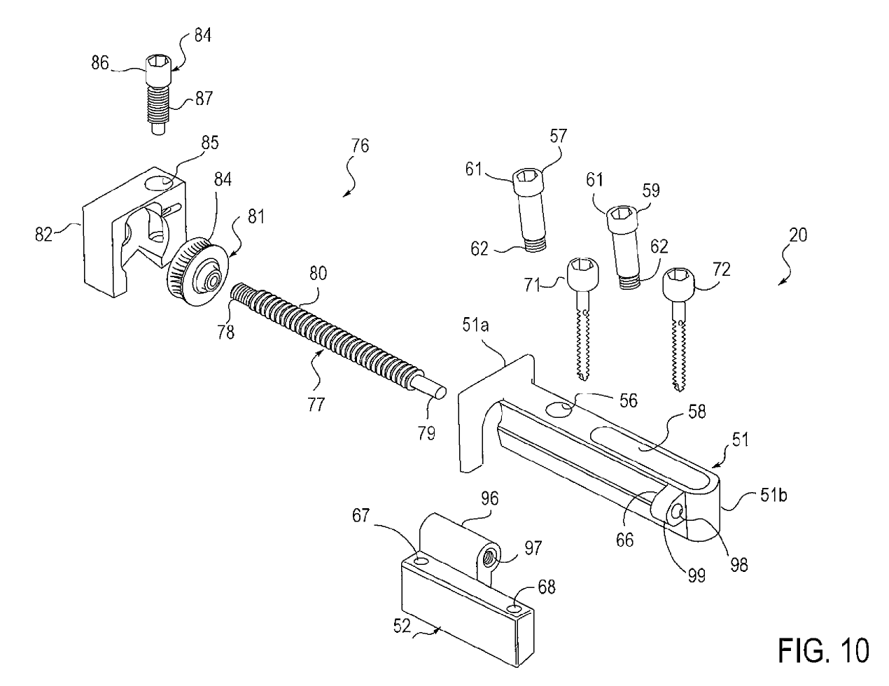

Generally, as shown in FIGS. 5-9, the bone reduction device 20 has a head 46 and a shaft 47 extending therefrom and a first or proximal end 48 and a second or distal end 49. The reduction device 20 includes an attachment 51 to the bone plate and an attachment 52 to the bone, which attachments are capable of movement relative to one another using the bone reduction device. The bone reduction device 20 includes a first member or a longitudinal bone plate attachment mechanism or arm 51 having at least one aperture or receptor for receipt of a bone plate attachment device such as a screw. More preferably, the bone plate attachment mechanism 51 has a first or proximal end 51a and a second or distal end 51b and includes a plurality of receptors or apertures on an elongate member or a longitudinally-extending arm 53, and more preferably) the bone plate attachment mechanism 51 includes two bone plate receptors or apertures spaced apart along a longitudinal axis 54 of the bone plate attachment arm 51. In the embodiment of FIGS. 1-10, the bone plate attachment arm 51 includes a first receptor or aperture or throughbore 56 adapted for receipt of a suitable attachment device 57 and a second aperture 58 adapted for receipt of a suitable attachment device 59. The bone attachment device(s) couple the bone plate attachment arm 51, by means of bone plate 26, to a segment 38 of the bone 21. The second aperture 58 has a longitudinal width greater than the width of the attachment device 59 which permits the bone plate attachment arm 51 to attach to a plurality of distinct bone plates 26 having threaded apertures 36 that are spaced along the longitudinal length of the shaft 28 at differing distances. Each of the attachment devices 57 and 59 preferably comprises a peg or bolt or screw, and more preferably a peg having a tool engaging head 61 and a threaded portion 62 spaced from the head (see FIG. 10). The threaded portion of the peg has an outer thread that mates with the inner thread of one or more threaded throughbores 36 in the bone plate 26. When in use, the pegs 57 and 59 extend through the plate attachment arm 51 and lock the bone reduction device 20 in position on the bone plate 26 by the attachment of the pegs 57 and 59 to the threaded aperture 36 of the bone plate. In the illustrated embodiment, the first and second pegs 57 and 59 are positioned in the first and second apertures 56 and 58 of the plate attachment arm 51 and in corresponding positioned threaded apertures or bores 36 in the plate 26.

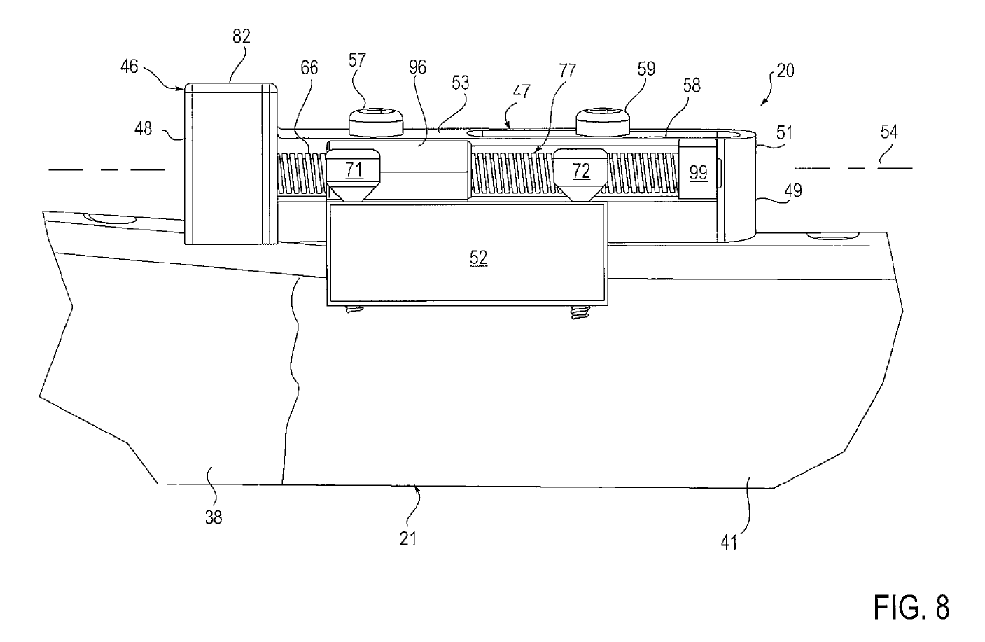

The bone reduction device 20 also includes a bone attachment arm or second member 52 positioned adjacent the plate attachment arm or first member 51 (see FIG. 8). Preferably, the bone attachment arm 52 is positioned so that it may slide along the plate attachment arm 51 and so that it may be guided and stabilized by the plate attachment arm 51. In this regard a portion of the bone attachment arm may be positioned within a longitudinal channel 66 on the plate attachment arm 51. The bone attachment arm 52 is thus positioned or disposed along side the plate attachment arm 51 and shaft 47 of the bone reduction device 20 and the shaft 28 of the bone plate 26. The bone attachment arm 52 preferably extends parallel to the plate attachment arm 51. The bone attachment arm 52 is separable from and capable of movement independent of the plate attachment arm 51 and has at least one throughbore or aperture for receipt of a bone attachment device. More preferably, the bone attachment arm 52 has a plurality of apertures or throughbores or apertures each for receipt of a plurality of bone attachment devices and more preferably, the bone attachment arm includes first and second throughbores 67 and 68 for receipt of respective first and second bone attachment devices 71 and 72. In position in the illustrated embodiment, the bone attachment arm includes first and second bone screws 67 and 68 extending through respective first and second apertures 71 and 72 and into bone 21. Preferably, each bone attachment device 67 and 68 is a bone screw or threaded bone screw for locking the bone attachment arm 52 in place on the bone 21. The bone screw may be any commercially available bone screw available from a variety of manufacturers.

Movement of the bone attachment arm 52 relative to the plate attachment arm 51 is facilitated by an adjustment mechanism 76 coupled between the bone attachment arm and plate attachment arm (see FIG. 10). The adjustment mechanism may include a metering screw assembly 76 that includes a metering screw 77 having first end or proximal end 78, a second end or distal end 79 and a intermediate or threaded portion 80. The proximal end 78 of the metering screw carries a knob or wheel 81. The wheel is contained within a housing 82 attached to the proximal end 51a of the plate attachment arm 51. The wheel 81 has an outer ridged surface 83 for engaging a metering nut 84 rotatably received within an aperture 85 provided in the top of the housing 82. The rotatable nut or metering nut 84 engages the wheel 81, and preferably the outer surface 83 of the wheel, to affect rotation of the wheel 81 and metering screw 77. Specifically, the metering nut 84 includes a head 86 for engaging a tool (not shown) and an intermediate threaded portion 87 for engaging the wheel 81. The distal end 79 of the metering screw is carried by a receptor 96 on the bone attachment arm 52 so that the metering screw 77 extends parallel to the bone attachment arm 52 and the plate attachment arm 51, and preferably along a side of each thereof. The intermediate threaded portion 80 of the metering screw 77 is received or engaged by an inner thread 97 on the metering screw receptor 96 carried by the bone attachment arm 52. The distal end 79 of the metering screw is rotatably captured within a bore 98 provided within a bracket or receptor 99 provided at the distal end 51b of plate attachment arm 51. Housing 82 and receptor 99 provide the respective proximal and distal end walls of longitudinal channel 66 in which the metering screw 77 is disposed. Rotation of the metering screw 77 causes the bone attachment arm 52 to travel proximally or distally along the length of the screw 77, thereby translating the bone attachment arm 52 relative to the plate attachment arm 51.

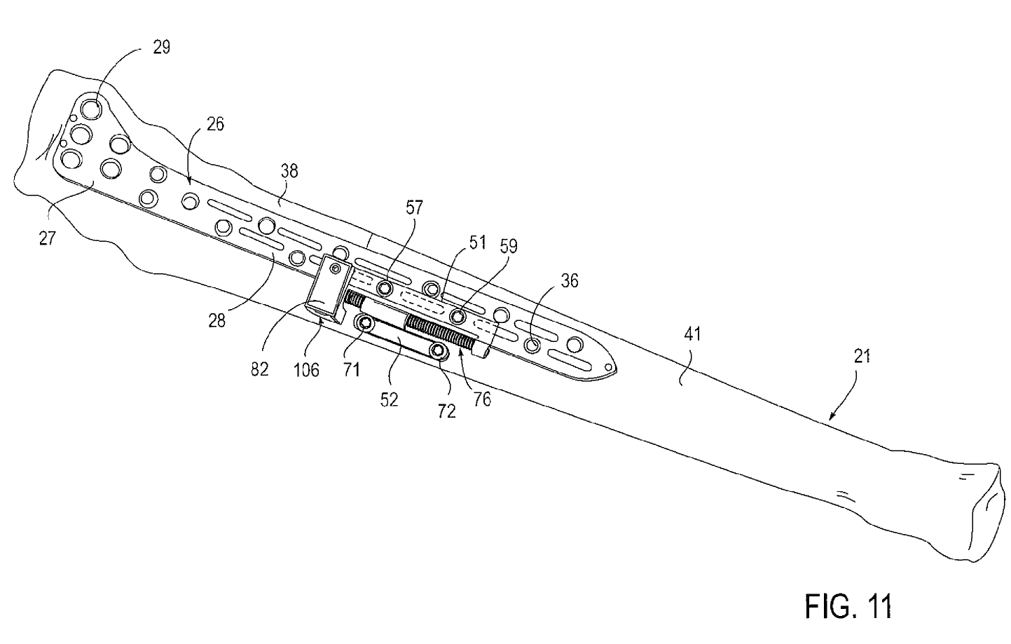

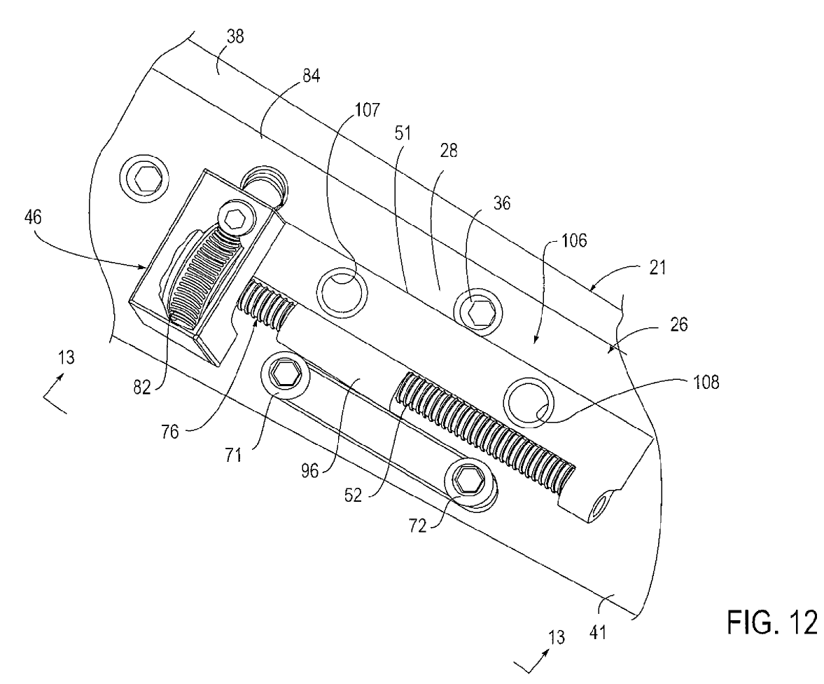

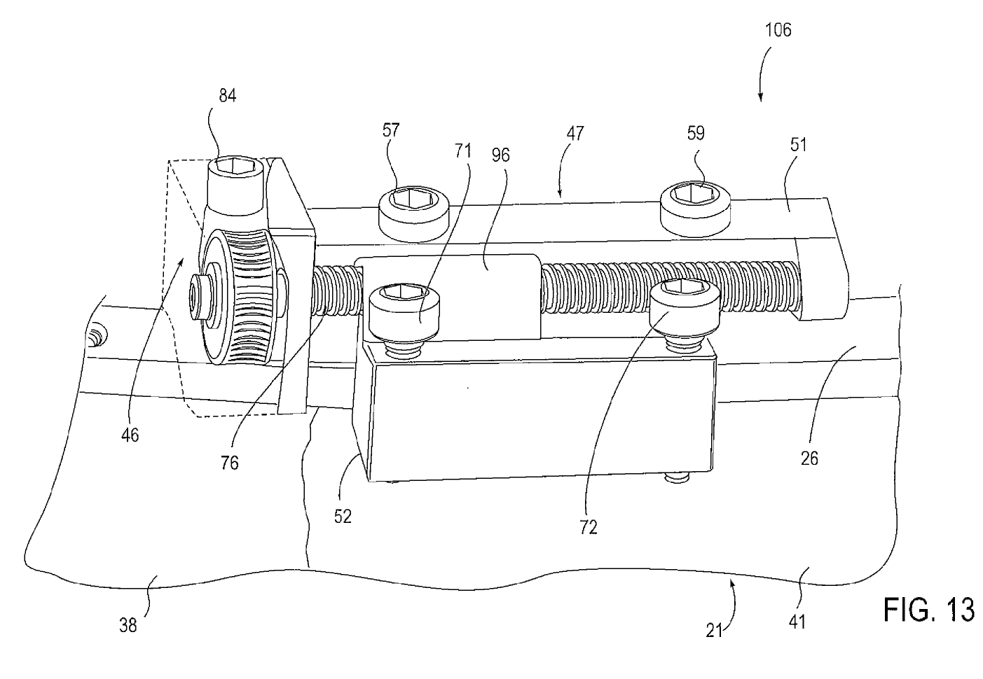

In an alternative embodiment, as illustrated in FIGS. 11-13, a bone reduction device 106 substantially similar to reduction device 20 is provided and like reference numerals have been used to describe like components of devices 20 and 106. The plate attachment arm 51 of reduction device 106 includes first and second apertures 107 and 108 for receipt of respective first and second attachment devices 57 and 59. In this embodiment, each aperture 107 and 108 is sized to correspond with the width or diameter of the respective attachment device or peg 57 and 59. Preferably, the first and second apertures 107 and 18 of the plate attachment arm 51 illustrated in FIGS. 11-13 are positioned to correspond with identically spaced apertures 36 on the bone plate 26.

In one preferred method of the invention described for example with respect to bone reduction device 20, an incision, for example three to four centimeters long, is made near the proximal portion of the bone 21. As discussed above, bone 21 has a proximal bone segment 38 and a distal bone segment 41. The proximal segment 38 includes a proximal end 111 and a distal end 112 and the distal segment 41 includes a proximal end 113 and a distal end 114 (see FIGS. 1-2). The bone plate 26 is inserted percutaneously into the body of the patient and fixed to the proximal piece 38 of bone, for example by screws 31, in a position allowing the operating physician to control the bone 21. A threaded bore bone plate 26 as described herein is provided which is first attached to the proximal portion or segment 38 of the bone by the threaded insertion of one or more bone screws 31 through threaded apertures 36 in the proximal portion 37 of the bone plate, locking the bone plate 26 to the proximal bone segment 38.

The bone reduction device 20 is attached to one or more threaded bore holes 36 in the bone plate 26 by rotatably inserting the pegs 57 and 59 or threaded portion 62 of the pegs through the apertures 56 and 58 in the plate attachment arm 51 and into the bone plate threaded bores 36. The bone reduction device 20 may be attached to the bone plate 26 before or after insertion into the body. The plate attachment arm 51 is attached to the bone plate 26 so that its proximal end 51 a is located on or near the distal end 112 of the proximal bone segment 38.

The metering screw assembly 76 is adjusted by rotation of the metering nut 84 so that the bone attachment arm 52 is positioned over the distal bone segment 41, and preferably positioned over or near the proximal portion 113 of the distal bone segment 41. In the illustrated embodiment, shown in FIGS. 1 and 3, the bone attachment arm 52 is moved distally into position for attachment to the distal bone segment 41. Once the plate attachment arm 51 is secured to the plate 26 and the bone attachment arm 52 is properly positioned, the bone attachment arm 52 is secured to the bone 21 adjacent to the plate 26 by one or more bone screws. Specifically, the bone attachment arm 52 is attached to the distal segment 41 of bone by insertion of bone screws 71 and 72 through the aperture(s) 67 and 68 of the bone attachment arm 52 and into bone 21. A screw driver or other tool (not shown) is then used to rotate the screw or metering nut 84 in the housing 82 of the bone reduction device 20. The metering nut rotation causes rotation of the wheel or knob 81, which in turn rotates the metering screw 77 attached thereto. Rotation of the metering screw 77 causes the bone attachment arm 52 to travel proximally or distally along the metering screw 77 by the threaded engagement of the threaded portion 80 of the metering screw 77 and the inner thread 97 of the metering screw receptor 96 on the bone attachment arm 52. The bone attachment arm or second member 52 also moves alongside the shaft 47 of the bone reduction device 20 relative to the plate attachment arm or first member 51 of the bone reduction device. The travel of the bone attachment arm 52 causes displacement or migration of the attached bone segment 41. More specifically, the distal bone segment 41 moves relative to the bone plate 26. As a result, the two fragments 38 and 41 of bone are brought into the desired alignment either by compression, namely, shortening the distance between the bone fragments 38 and 41, or distraction, lengthening the distance between the bone fragments 38 and 41. In the illustrated embodiment, shown in FIGS. 2 and 4, the distal bone fragment 41 is moved proximally and aligned with the proximal bone segment 38.

Once in the proper anatomical position to heal, the second or distal bone segment 41 may then be secured or locked to the bone plate 26 by the insertion of bone screws 31 through apertures 36 and/or bushings 32 provided in the bone plate 26 and into the bone 21.

Advantageously, the use of bone screws and a locking bone plate, permits the use of a very small incision to attach and use the bone reduction device of the present invention, which provides advantages to both the physician and the patient. For instance, contrary to commercially available devices, the bone reduction device disclosed herein avoids the need to use a device positioned at the end of a bone plate, which necessitates the lengthening an incision and an increase in devascularization of the bone. Additionally, the bone reduction device which attaches to a locking bone plate avoids the need to place a screw at the end of the bone in order to use the device, which often times requires a hole to be drilled either distal or proximal to the bone plate causing a stress riser in the bone which stress riser is not shielded by the plate bone construct, leading to an increased risk of the bone breaking at that empty bone screw hole. The use of a bone plate as described herein has threaded bores that permit use of locking bone screw and which permit use of a reduction device as disclosed that attaches to one or more threaded bore holes in the bone plate. The bone reduction device provides an improved reduction device wherein the device is used during open reduction and internal fixation of bones to achieve reduction of the bone by moving the locking bone plate relative to the bone.

While the embodiments described refer to specific attachment to the bone segments, variations may be made without departing from the overall scope of the present invention. For example, it is contemplated that the device may be reversed in orientation from that specifically described herein.

Although various representative embodiments of this invention have been described above with a certain degree of particularity, those skilled in the art could make numerous alterations to the disclosed embodiments without departing from the spirit or scope of the inventive subject matter set forth in the specification and claims. All directional references (e.g., upper, lower, upward, downward, left, right, leftward, rightward, top, bottom, above, below, vertical, horizontal, clockwise, counterclockwise, x-axis, y-axis, and z-axis) are only used for identification purposes to aid the reader's understanding of the embodiments of the present invention, and do not create limitations, particularly as to the position, orientation, or use of the invention unless specifically set forth in the claims. Joinder references (e.g., attached, coupled, connected) are to be construed broadly and may include intermediate members between a connection of elements and relative movement between elements. As such, joinder references do not necessarily infer that two elements are directly connected and in fixed relation to each other.

In some instances, components are descried with reference to “ends” having a particular characteristic and/or being connected with another part. However, those skilled in the art will recognize that the present invention is not limited to components which terminate immediately beyond their points of connection with other parts. Thus, the term “end” should be interpreted broadly, in a manner that includes areas adjacent, rearward, forward of, or otherwise near the terminus of a particular element, link, component, part, member. In methodologies directly or indirectly set forth herein, various steps and operations are described in one possible order of operation, but those skilled in the art will recognize that steps and operations may be rearranged, replaced, or eliminated without necessarily departing from the spirit and scope of the present invention. It is intended that all matter contained in the above description or shown in the accompanying drawings shall be interpreted as illustrative only and not limiting. Changes in detail or structure may be made without departing from the spirit of the invention as defined in the appended claims.

Although the present invention has been described with reference to preferred embodiments, persons skilled in the art will recognize that changes may be made in form and detail without departing from the spirit and scope of the invention.

What is claimed is:

1. A bone reduction device for use with a bone plate having a head and shaft and at least one plate attachment device and at least one bone attachment device to repair a segmented bone, comprising a plate attachment arm having at least one aperture adapted to receive the at least one plate attachment device for coupling the plate attachment arm to the shaft of the bone plate, a bone attachment arm disposed alongside the plate attachment device and having at least one aperture adapted to receive the at least one bone attachment device for coupling the bone attachment arm to a segment of the bone, the bone attachment arm being moveable relative to the plate attachment arm, an adjustment mechanism carried by the plate attachment arm and engaging the bone attachment arm for translating the bone attachment arm relative to the plate attachment arm.

2. The bone reduction device of claim 1 wherein the adjustment mechanism is a metering screw assembly.

3. A bone reduction device for use with a bone plate having a head and shaft to repair a bone having first and second bone segments, comprising a first member adapted to couple to the shaft of the bone plate so as to be coupled to the first bone segment when the bone plate is coupled to the first bone segment, a second member disposed alongside the first member and being moveable relative to the first member and adapted to couple to the second bone segment, the second member extending alongside the shaft of the bone plate when the first member is coupled to the shaft of the bone plate, and an adjustment mechanism coupled between the first member and the second member for moving the second member relative to the member so as to move the second bone segment into close proximity to the first bone segment and facilitate coupling the bone plate to the second bone segment.

4. The bone reduction device of claim 3 wherein the first member is a first elongate member and the second member extends parallel to the first member.

5. A method of reducing a segmented bone comprising attaching a bone plate having a head and shaft to a first bone segment, attaching a first member of a bone reduction device to the bone plate, attaching a second member of the bone reduction device to a second bone segment and repositioning the second bone segment relative to the first bone segment by moving the second member alongside the shaft of the bone reduction device relative to the first member of the bone reduction device.

6. The method of claim 5 wherein the first member comprises a plate attachment arm and attachment of the first member of the bone reduction device to the bone plate includes inserting an attachment device through an aperture in the plate attachment arm of the bone repair device and fastening the attachment device to the bone plate.

7. The method of claim 5 wherein the second member comprises a bone attachment arm and attachment of the second member of the bone reduction device to the second bone segment includes insertion of a bone screw through an aperture in the bone attachment arm of the bone repair device and into the second bone segment.

8. The method of claim 5 wherein repositioning the second bone segment relative to the first bone segment includes actuating a metering screw assembly carried by the first member of the bone reduction device operably engaged with the second member so as to move the second member and second bone segment relative to the first bone segment and first member to align the first bone segment and second bone segment adjacent the bone plate.
